(19) United States
(12) Patent Application Publication
(10) Pub. No.: US 2021/0177869 A1
(43) Pub. Date: Jun. 17, 2021

(54) METHODS AND COMBINATIONS FOR MODULATING TOLERANCE TO OPIATES, OPIOIDS OR OPIOID ANALGESICS AND TREATING ACUTE AND CHRONIC PAIN

(71) Applicant: CHINA MEDICAL UNIVERSITY, Taichung (TW)

(72) Inventor: Chia-Hung HSIEH, Taichung (TW)

(21) Appl. No.: 17/267,775

(22) PCT Filed: Aug. 9, 2019
(86) PCT No.: PCT/CN2019/100066

§ 371 Date: Feb. 10, 2021

Related U.S. Application Data
(60) Provisional application No. 62/717,774, filed on Aug. 11, 2018.

(51) Int. Cl. A61K 31/635; A61K 31/55; A61P 25/36

(52) U.S. Cl. CPC A61K 31/635; A61P 25/36; A61K 31/55

(57) ABSTRACT

The present disclosure provides methods for modulating tolerance to opiates, opioids or opioid analgesics and/or treating acute and chronic pain and/or decreasing opiate or opioid-induced side effects. Also provided is a combination comprising a therapeutically effective amount, or a sub-therapeutically effective amount, of opiate, opioid or opioid analgesic and a therapeutically effective amount of a cystine/glutamate antiporter system x_{c}⁻ inhibitor.

### FIELD OF THE INVENTION

The present disclosure relates generally to a field of pain treatment and opioid tolerance reduction. In particular, the present disclosure relates to methods and combination for modulating tolerance to opiates, opioids or opioid analgesics and treating acute and chronic pain.

### BACKGROUND OF THE INVENTION

Opioids or opioid analgesics are the most effective treatment for pain compared to other pain relievers. Unfortunately, the development of tolerance, dependence, addiction or other side effects such as sedation, dizziness, nausea, vomiting, constipation and respiratory depression during chronic opioid use further limit the clinical utility of these drugs. These side effects also cause the opioid crisis which affects the health, social, and economic welfare of all societies in many countries, such as United States and China, due to the widespread use of prescription and non-prescription opioid drugs. For many years, efforts to overcome the side effects of opioids have met with limited success. All of opioids that produce analgesia also can cause tolerance, addiction and withdrawal. Despite a variety of in vitro and in vivo studies provide a solid framework for translational research contributing to developments in opioid therapeutics that may reduce the severity of one or more of these side effects, there is still no curable medication for stopping opioid-induced tolerance and addiction and therefore, addressing related issues continues to be global unmet medical needs.

Opioid tolerance is characterized by a reduced responsiveness to an opioid agonist and is usually manifest by the need to use increasing doses to achieve the desired effect for long-term use of opioids. The development and extent of tolerance are dependent on the drug interactions with the opioid receptors, dose, and frequency of administration. A lot of studies reported several mechanisms involved in opioid tolerance at a behavioral level. These mechanisms are upregulation of drug metabolism, desensitization of receptor signaling, and downregulation of receptors, as well as the initiation of compensatory/opponent processes (Kest, B., et al., Naloxone precipitated withdrawal jumping in 11 inbred mouse strains: evidence for common genetic mechanisms in acute and chronic morphine physical dependence. Neuroscience, 2002. 115(2): p. 463-9). Due to tolerance, patients who use opioids must often increase their dose to keep pain relief. That, however, increases the risk of opioid-induced side effects especially in overdose.

US 20180193331 discloses a method of treating or preventing an opioid induced adverse pharmacodynamic response comprising administering to a patient in need thereof an effective amount of buprenorphine.

US 20150258108 provides A method for modulating tolerance to an opioid analgesic in a patient undergoing opioid analgesic therapy, the method comprising interrupting or administering concurrently with said opioid analgesic therapy an amount of noribogaine, noribogaine derivative, or pharmaceutically acceptable salt and/or solvate thereof.

US 20180362607 provides a method of treating morphine tolerance and/or symptoms associated therewith by administration to a subject in need thereof a DN-TNF polypeptide that inhibits the activity of soluble TNF-but not transmembrane TNF-alpha.

Effective strategies to modulate opioid tolerance have not been discovered despite the extensive efforts devoted to this area of research. Therefore, there is a need in the art for methods and drugs for countering opioid tolerance.

### SUMMARY OF THE INVENTION

Disclosed in the present disclosure is a method for modulating tolerance to opiates, opioids or opioid analgesics in a subject who has developed or is at risk of developing a tolerance for the opioid or analgesic, comprising administering a therapeutically effective amount, or a sub-therapeutically effective amount, of opiate, opioid or opioid analgesic and a therapeutically effective amount of a cystine/glutamate antiporter system x_{c}⁻ inhibitor.

In some embodiments, the modulating tolerance includes delaying, reducing, relieving, attenuating, and/or reversing tolerance to opioids or opioid analgesics.

In some embodiments, the subject obtains therapeutic effect from a lower dose of the opiate, opioid or opioid analgesic than before modulation of tolerance. In one aspect, the modulated subject obtains an improved therapeutic effect from the same dose of the opioid or opioid analgesic compared to before modulation.

In one embodiment, the subject exhibits opiate, opioid or opioid analgesic tolerance prior to said administration and reduced opioid or opioid analgesic tolerance following said administration.

Also disclosed in the present disclosure is a method for treating acute and chronic pain and/or decreasing opiate or opioid-induced side effects in a subject in a need of opiate, opioid or opioid analgesic therapy, comprising administering a therapeutically effective amount, or a sub-therapeutically effective amount, of opiate, opioid or opioid analgesic and a therapeutically effective amount of a cystine/glutamate antiporter system x_{c}⁻ inhibitor.

In one embodiment, the cystine/glutamate antiporter system x_{c}⁻ inhibitor enhances the analgesic effect of opiate, opioid or opioid analgesic.

The opiate or opioid-induced side effects include, but are not limited to, sedation, dizziness, bowel dysfunction (such as constipation, decreased gastric emptying, abdominal cramping, spasm, bloating, delayed gastro-intestinal transit and formation of hard dry stools), nausea, vomiting, somnolence, physical dependence, tolerance, addiction, respiratory depression, headache, dry mouth, sweats, asthenia, hypotension, dysphoria, delirium, miosis, pruritis, urticaria, urinary retention, hyperalgesia and allodynia and a combination thereof.

In some embodiments of the methods of the present disclosure, the cystine/glutamate antiporter system x_{c}⁻ inhibitor is administered concurrently or separately with an opiate, opioid or opioid analgesic. In one embodiment, the amount of the opiate, opioid or opioid analgesic can be reduced when administering concurrently or separately with the cystine/glutamate antiporter system x_{c}⁻ inhibitor. In one embodiment, during the concurrent administration, the amount of the opiate, opioid or opioid analgesic is reduced.

In one embodiment of the methods of the present disclosure, the therapeutically effective amount of opiate, opioid or opioid analgesic is depended on the dosages used clinically; for example, clinical dosages stated in the package insert of medication.

In one embodiment of the methods of the present disclosure, the sub-therapeutically effective amount of opiate, opioid or opioid analgesic is under the dosage used clinically; for example, under the clinical dosage stated in the package insert of medication.

In one embodiment of the methods of the present disclosure, the therapeutically effective amount of a cystine/glutamate antiporter system x_{c}⁻ inhibitor is depended on the dosages used clinically. In a further embodiment, the amount of sulfasalazine ranges from about 0.75 mg/kg to about 28.57 mg/kg.

Also disclosed in the present disclosure is a combination comprising a therapeutically effective amount, or a sub-therapeutically effective amount, of opiate, opioid or opioid analgesic and a therapeutically effective amount of a cystine/glutamate antiporter system x_{c}⁻ inhibitor. In one embodiment, the opiate, opioid or opioid analgesic and the cystine/glutamate antiporter system x_{c}⁻ inhibitor are contained in a medicament or the opiate, opioid or opioid analgesic and the cystine/glutamate antiporter system x_{c}⁻ inhibitor are each independently contained in a separate medicament.

In some embodiments in the methods and combination described herein, the opiate, opioid or opioid analgesic include, but are not limited to, opiate, opioid, codeine, fentynal, hydrocodone, hydromorphone, buprenorphine, thebaine, meperidine, methadone, morphine, oxycodone, oxycodone, acetaminophen, oxycodone and naloxone, heroin, heroin laced with fetynal, pethidine, opium, NKTR-181, Difelikefalin, tramadol, tapentadol, levorphanol, sufentanil, pentazocine, and oxymorphone and a combination thereof.

In some embodiments in the methods and combination described herein, the cystine/glutamate antiporter system x_{c}⁻ inhibitor include, but are not limited to, sorafenib, regorafenib, sulfasalazine, 2-hydroxy-5-((4-(N-pyridin-2-ylsulfamoyl)phenyl)ethynyl)benzoic acid, 5-aminosalicylic acid (5-ASA), sulfapyridine (SP), erastin, L-glutamate, L-cystine, L-alpha-aminoadipate, L-alpha-aminopimelate, L-homocysteate, L-b-N-oxalyl-L-a,b-diaminopropionate (beta-L-ODAP), L-alanosine, ibotenate, L-serine-O-sulphate, (RS)-4-bromohomoibotenate, quisqualate, (S)-4-carboxyphenylglycine, RS-4-Br-Homo-IBO, 2-amino-3-(5-methyl-3-oxo-1,2-oxazol-4-yl)propanoic acid (AMPA), arachidonylcyclopropylamide (ACPA), N-acetylamino-3-chloro-N-(2-diethylaminoethyl)benzamide (NACPA), TFMIH, NEIH, (S)-4-carboxyphenyglycine (4-S-CPG), 4-S-SPG, TSA, CPPG and capsazepine and any combination thereof. In some embodiments, the cystine/glutamate antiporter system x_{c}⁻ inhibitor is sorafenib, regorafenib, sulfasalazine or capsazepine or a combination thereof. In some embodiments, the cystine/glutamate antiporter system x_{c}⁻ inhibitor is sulfasalazine or capsazepine.

### BRIEF DESCRIPTION OF THE DRAWINGS

FIGS. 1 A to F show that knockout of system x_{c}⁻ in mice is able to increase the analgesic effect of opioid. (A and B) Behavioral avoidance in the hot plate test and behavior withdraw in the Von-Frey test for system x_{c}⁻ knockout (xCT KO) mice and wild-type (WT) mice. (C and D) Behavioral avoidance in the hot plate test and behavior withdraw in the Von-Frey test for system x_{c}⁻ knockout (xCT KO) mice and wild-type (WT) mice received intraperitoneal injection of morphine (10 mg/kg/day) for 7 days. (C and D) Behavioral avoidance in the hot plate test and behavior withdraw in the Von-Frey test for system x_{c}⁻ knockout (xCT KO) mice and wild-type (WT) mice received intraperitoneal injection of methadone (4 mg/kg/day) for 7 days. 6-8 animals for each group. *P<0.0001 compared to WT mice, two-tailed unpaired Student's t-test.

FIGS. 2 A to D show that the pharmacological inhibition of system x_{c}⁻ enhances the analgesic effect of opioid. (A and B) Behavioral avoidance in the hot plate test and behavior withdraw in the Von-Frey test for in mice with vehicle, sulfasalazine (SSZ, 30 mg/kg/day), morphine (10 mg/kg/day) or the combination of SSZ and morphine treatment for 8 days. (C and D) Behavioral avoidance in the hot plate test and behavior withdraw in the Von-Frey test for in mice with vehicle, sulfasalazine (SSZ, 30 mg/kg/day), methadone (5 mg/kg/day) or the combination of SSZ and methadone treatment for 8 days. 6-8 animals for each group. *P<0.001 compared to vehicle, Two-way ANOVA followed by Bonferroni test.

FIGS. 3 A to C show that the blockage of system x_{c}⁻ delays the opioid tolerance. (A) the time response curves of morphine-induced analgesic effect in system x_{c}⁻ knockout (xCT KO) mice and wild-type (WT) mice received the morphine (20 mg/kg/day) twice daily for 7 days. (B and C) the time response curves of morphine-induced analgesic effect in WT mice received the sulfasalazine (SSZ, 60 mg/kg/day), morphine (20 mg/kg/day), capsazepine (10 mg/kg/day) or their combinations twice daily for 7 days. 6-8 animals for each group.

FIGS. 4 A and B show that the blockage of system x_{c}⁻ inhibits opioid dependence and improves the withdrawal symptoms. (A) the jumping behavior in system x_{c}⁻ knockout (xCT KO) mice and wild-type (WT) mice with chronic morphine treatment and naloxone-induced acute withdrawal syndromes. (B) the jumping behavior in WT mice with chronic morphine dependence and received vehicle, sulfasalazine (SSZ, 30 mg/kg), capsazepine (5 mg/kg), morphine (10 mg/kg) or their combinations before naloxone-induced acute withdrawal syndromes. 6-8 animals for each group. *P<0.0001 compared to vehicle, #P<0.0001 compared to morphine alone, one-way ANOVA followed by Tukey test.

FIG. 5 shows that the system x_{c}⁻ inhibitor, SSZ, improves the opioid-induced constipation. The changes in the stool weight for wild-type mice received vehicle, sulfasalazine (SSZ, 30 mg/kg), morphine (10 mg/kg) or the combination of morphine and SSZ for 7 days. 6-8 animals for each group. *P<0.001 compared to vehicle, Two-way ANOVA followed by Bonferroni test.

### DETAILED DESCRIPTION OF THE INVENTION

As used in this specification and the appended claims, the singular forms “a,” “an” and “the” include plural references unless the content clearly dictates otherwise.

As used herein, the terms “treating” or “treatment of” a disease state includes: 1) inhibiting the disease state, i.e., arresting the development of the disease state or its clinical symptoms; 2) or relieving the disease state, i.e., causing temporary or permanent regression of the disease state or its clinical symptoms.

As used herein, the term “therapeutically effective amount” refers to the amount of an agent described herein that is sufficient to effect treatment when administered to a patient in need of such treatment. The therapeutically effective amount will vary depending upon the specific activity of the therapeutic agent being used, and the age, physical condition, existence of other disease states, and nutritional status of the patient.

As used herein, the term “sub-therapeutically effective amount” refers to the amount of an agent described herein that is under the dosage used clinically. For example, the amount is under the dosage in the package insert of medications. A sub-therapeutically effective amount dose does not preclude that the amount can have other therapeutic, prophylactic or pharmacodynamics effects.

As used herein, the term “system x_{c}⁻” refers to an amino acid antiporter that typically mediates the exchange of extracellular L-cystine and intracellular L-glutamate across the cellular plasma membrane.

As used herein, the term “tolerance” refers to the psychological and/or physiologic process wherein the subject adjusts to the frequent presence of a substance such that a higher dose of the substance is required to achieve the same effect. Tolerance may develop at different times for different effects of the same drug.

As used herein, the term “subject” includes both human and veterinary subjects, for example, humans, non-human primates, dogs, cats, horses, rats, mice, and cows. Similarly, the term mammal includes both human and non-human mammals. In some embodiments, a subject is a patient, such as patient prescribed one or more opioid medications.

As used herein, the term “opioid analgesic” means any material that produces an analgesic effect through modulation of an opioid receptor. The term includes all pharmaceutically active forms of the opioid analgesic, including the free base form of the agent, and all pharmaceutically acceptable salts, complexes, crystalline forms, co-crystals, hydrates, solvates, and mixtures thereof, where the form is pharmaceutically active.

As used herein, the term “opioid-induced side effect” means an unintended side effect experienced by a patient receiving opioid therapy for an intended therapeutic effect. Typically, the intended affect is analgesia.

Drug tolerance to opioids or opioid analgesics is common and may be psychological and/or physiological. A patient who has developed tolerance to the opioids is not necessarily depended or addicted to or misusing the analgesic. Drug tolerance occurs when the patient's reaction to the drug is reduced, requiring an increase in dose to achieve the same desired effect. Drug tolerance requires that the dosage of analgesic be increased in order to provide sustained analgesic effect. However, high doses of opioids may lead to serious complications and side effects.

The present disclosure unexpectedly found that a cystine/glutamate antiporter system x_{c}⁻ inhibitor can modulate tolerance to opiates, opioids or opioid analgesics. The present disclosure also unexpectedly found that a cystine/glutamate antiporter system x_{c}⁻ inhibitor can enhance the analgesic effect of opiate, opioid or opioid analgesic and/or decrease opiate or opioid-induced side effects. Accordingly, the present disclosure provides methods for modulating tolerance to opiates, opioids or opioid analgesics and/or treating acute and chronic pain and/or decreasing opiate or opioid-induced side effects. Also provided is a combination comprising a therapeutically effective amount, or a sub-therapeutically effective amount, of opiate, opioid or opioid analgesic and a therapeutically effective amount of a cystine/glutamate antiporter system x_{c}⁻ inhibitor.

In one aspect, the present disclosure provides methods for modulating tolerance to opiates, opioids or opioid analgesics in a subject who has developed or is at risk of developing a tolerance for the opioid or analgesic, comprising administering a therapeutically effective amount, or a sub-therapeutically effective amount, of opiate, opioid or opioid analgesic and a therapeutically effective amount of a cystine/glutamate antiporter system x_{c}⁻ inhibitor.

The modulation tolerance includes, but is not limited to, delaying, reducing, relieving, attenuating, and/or reversing tolerance to opioids or opioid analgesics. The subject can obtain therapeutic effect from a lower dose of the opiate, opioid or opioid analgesic than before modulation of tolerance. In one embodiment, the modulated subject obtains an improved therapeutic effect from the same dose of the opioid or opioid analgesic compared to before modulation. Also, the subject exhibits opiate, opioid or opioid analgesic tolerance prior to said administration and reduced opioid or opioid analgesic tolerance following said administration.

In another aspect, the present disclosure provides methods for treating acute and chronic pain and decreasing opiate or opioid-induced side effects in a subject, comprising administering a therapeutically effective amount, or a sub-therapeutically effective amount, of opiate, opioid or opioid analgesic and a therapeutically effective amount of a cystine/glutamate antiporter system x_{c}⁻ inhibitor.

The present disclosure also provides a combination comprising a therapeutically effective amount, or a sub-therapeutically effective amount, of opiate, opioid or opioid analgesic and a therapeutically effective amount of a cystine/glutamate antiporter system x_{c}⁻ inhibitor. The opiate, opioid or opioid analgesic and a therapeutically effective amount of a cystine/glutamate antiporter system x_{c}⁻ inhibitor can be contained in a medicament or each independently contained in a separate medicament.

In certain embodiments, the opioid-induced side effect can be caused by the administration of opiate, opioid or opioid analgesic. The opioid-induced side effect includes, but in not limited to, sedation, dizziness, bowel dysfunction (such as constipation, decreased gastric emptying, abdominal cramping, spasm, bloating, delayed gastro-intestinal transit, formation of hard dry stools), nausea, vomiting, somnolence, physical dependence, tolerance, addiction, respiratory depression, headache, dry mouth, sweats, asthenia, hypotension, dysphoria, delirium, miosis, pruritis, urticaria, urinary retention, hyperalgesia and allodynia.

In certain embodiments, an opiate, opioid or opioid analgesic is administered concurrently with and a cystine/glutamate antiporter system xc-inhibitor. In one embodiment, the opiate, opioid or opioid analgesic is administered in a therapeutically effective amount, or a sub-therapeutically effective amount, to provide an analgesic effect. In one embodiment, the opiate, opioid or opioid analgesic is administered in a sub-therapeutically effective amount, yet still in an effective amount to provide an analgesic effect. In one embodiment, the cystine/glutamate antiporter system xc-inhibitor serves to modulating tolerance to opioids or opioid analgesics, or reduce, prevent, minimize, inhibit, ameliorate or reverse the opioid-induced side effect.

Examples of the opiate or opioid-induced side effects include, but are not limited to, sedation, dizziness, bowel dysfunction (such as constipation, decreased gastric emptying, abdominal cramping, spasm, bloating, delayed gastro-intestinal transit and formation of hard dry stools), nausea, vomiting, somnolence, physical dependence, tolerance, addiction, respiratory depression, headache, dry mouth, sweats, asthenia, hypotension, dysphoria, delirium, miosis, pruritis, urticaria, urinary retention, hyperalgesia and allodynia, and a combination thereof. The compounds for opiates, opioids or cystine/glutamate antiporter system x_{c}⁻ inhibitors are pharmaceutically acceptable salts thereof, pharmaceutically acceptable solvates thereof, and pharmaceutically acceptable salts solvated with pharmaceutically acceptable solvents thereof.

Examples of the opiate, opioid or opioid analgesic include, but are not limited to, opiate, opioid, codeine, fentynal, hydrocodone, hydromorphone, buprenorphine, thebaine, meperidine, methadone, morphine, oxycodone, oxycodone, acetaminophen, oxycodone and naloxone, heroin, heroin laced with fetynal, pethidine, opium, NKTR-181, Difelikefalin, tramadol, tapentadol, levorphanol, sufentanil, pentazocine, and oxymorphone and a combination thereof.

Examples of the cystine/glutamate antiporter system x_{c}⁻ inhibitor include, but are not limited to, sorafenib, regorafenib, sulfasalazine, 2-hydroxy-5-((4-(N-pyridin-2-ylsulfamoyl)phenyl)ethynyl)benzoic acid, 5-aminosalicylic acid (5-ASA), sulfapyridine (SP), erastin, L-glutamate, L-cystine, L-alpha-aminoadipate, L-alpha-aminopimelate, L-homocysteate, L-b-N-oxalyl-L-a,b-diaminopropionate (beta-L-ODAP), L-alanosine, ibotenate, L-serine-O-sulphate, (RS)-4-bromohomoibotenate, quisqualate, (S)-4-carboxyphenylglycine, RS-4-Br-Homo-IBO, 2-amino-3-(5-methyl-3-oxo-1,2-oxazol-4-yl)propanoic acid (AMPA), Arachidonylcyclopropylamide (ACPA), N-acetylamino-3-chloro-N-(2-diethylaminoethyl)benzamide (NACPA), TFMIH, NEIH, (S)-4-carboxyphenyglycine (4-S-CPG), 4-S-SPG, TSA, CPPG and capsazepine. In some embodiments, the cystine/glutamate antiporter system x_{c}⁻ inhibitor is sorafenib, regorafenib or sulfasalazine or capsazepine or a combination thereof.

The combination or medicament may be formulation as composition in various forms. For example, it can be a soft chew composition, powder, emulsion composition, aqueous composition, capsule, tablet or gel.

Administration of the opiate, opioid or opioid analgesic, the cystine/glutamate antiporter system x_{c}⁻ inhibitor or their combinations or medicaments according to the present disclosure may be via any common route so long as the target tissue is available via that route. This includes oral, nasal, or buccal. Alternatively, administration may be by parenteral, intradermal, subcutaneous, intramuscular, intraperitoneal or intravenous injection. In certain embodiments, the combinations or medicaments of the present disclosure are formulated for intravenous or subcutaneous or oral administration.

The opiate, opioid or opioid analgesic, the cystine/glutamate antiporter system x_{c}⁻ inhibitor or their combinations or medicaments according to the present disclosure may also be administered parenterally or intraperitoneally. By way of illustration, solutions of the opiate, opioid or opioid analgesic, the cystine/glutamate antiporter system x_{c}⁻ inhibitor or their combinations according to the present disclosure as free base or pharmacologically acceptable salts can be prepared in water suitably mixed with a surfactant. Dispersions can also be prepared in glycerol, liquid polyethylene glycols, and mixtures thereof and in oils. Under ordinary conditions of storage and use, these preparations generally contain a preservative to prevent the growth of microorganisms.

Formulations or medicaments suitable for parenteral administration include aqueous and non-aqueous sterile injection solutions which may contain antioxidants, buffers, bacteriostats and solutes which render the formulation isotonic with the blood of the intended recipient; and aqueous and non-aqueous sterile suspensions which may include suspending agents and thickening agents.

Formulations or medicaments suitable for oral administration may be presented as discrete units such as capsules, sachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be presented as a bolus, electuary or paste.

A tablet may be made by compression or moulding, optionally with one or more excipients or carriers. Compressed tablets may be prepared by compressing in a suitable machine the active ingredient in a free-flowing form such as a powder or granules, optionally mixed with a binder (e.g inert diluent, preservative disintegrant (e.g. sodium starch glycolate, cross-linked polyvinyl pyrrolidone, cross-linked sodium carboxymethyl cellulose) surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein using, for example, hydroxypropylmethyl cellulose in varying proportions to provide the desired release profile. Tablets may optionally be provided with an enteric coating.

Formulations or medicaments suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations or medicaments for rectal administration may be presented as a suppository with a suitable base comprising for example cocoa butter or a salicylate.

Formulations or medicaments suitable for intrapulmonary or nasal administration is administered by rapid inhalation through the nasal passage or by inhalation through the mouth so as to reach the alveolar sacs. Suitable formulations or medicaments include aqueous or oily solutions of the active ingredient. Formulations or medicaments suitable for aerosol or dry powder administration may be prepared according to conventional methods.

The present disclosure has been described in terms of particular embodiments as provided herein to comprise preferred modes for the practice of the disclosure. It will be appreciated by those of skill in the art that, in light of the present disclosure, numerous modifications and changes can be made in the particular embodiments exemplified without departing from the intended scope of the disclosure.

### EXAMPLE

Methods and Material

Thermal Hyperalgesia and Mechanical Allodynia

The thermal hyperalgesia was evaluated by placing the mice in a 1000 ml beaker and on the 56±2° C. hot plate with the maximum of 35 second to prevent tissue damage. The time is recorded when the mice jumps on to the edge of the beaker, the value is considered as the withdrawal threshold. A customized transparent acrylic chamber with the size of 20×20×20 cm and 5 mm thick acrylic with 2 mm diameter holes throughout the entire platform is used to determine the mechanical allodynia and thermal hyperalgesia. Mice were placed in the chamber to evaluate the mechanical allodynia by applying a 0.6 g pressure von Frey hair (Touch-Test Sensory Evaluator, North Coast Medical, Inc) to the hind paws of the mice for five times with a five second interval. If the mice hind paw is withdrawn from the platform over three times, the pressure of the von Frey hair will be recorded and regarded as the withdrawal threshold. On the contrary, if the mice did not show any difference, the pressure of the von Frey hair will increase and tested on the mice again.

Fecal Collection and Stool weighed

Fecal collections and the stool weighed were recorded before and after morphine and/or SSZ administration. The fecal was collected every two days.

Conditional Place Preference

Background CPP score was first tested by a shuttle box. In the 9 a.m. after the mice were intraperitoneally injected with PBS, they were placed in the dark chamber for 20 minutes separately; in the 2 p.m., after another injection of PBS, the mice were placed in the light chamber for 20 minutes, this process continued for 3 days. On the 4th day, wild type and knockout mice were randomly separated into control and experimental group respectively. Starting from the 4th day, the control group continued the process of injecting PBS at both 9 a.m. and 2 p.m.; as for the experimental group, mice were also injected PBS in the 9 a.m. and placed in the dark chamber for 20 minutes, however in the 2 p.m. mice were injected with morphine and/or system x_{c}⁻ inhibitor, SSZ or capsazepine, and placed in the light chamber for 20 minutes. On the 7 day, the morphine challenge is conducted by recording the time stayed in each chamber for 2.

Chronic Morphine Dependence

Chronic morphine dependence was induced by repeated injections for four consecutive days with an escalating dose schedule. Mice received morphine twice daily (morning and afternoon) for 4 days (day 1: 12 mg/kg and 25 mg/kg; day 2: 25 mg/kg and 50 mg/kg; day 3: 50 mg/kg and 75 mg/kg; day 4: 75 mg/kg and 100 mg/kg). On the testing day (day 5), a final morphine dose (50 mg/kg) was administered before exam.

Naloxone-Precipitated Withdrawal

A single dose of naloxone (5 mg/kg) was administered to all mice 60 mins after the final morphine dose. Immediately, following with naloxone injection, animals were placed into individual Plexiglas observation cylinders. Withdrawal jumping response symptoms were recorded and the frequency of jumps for each mouse was summed over one hour.

The Population-Based Cohort Study

Taiwan launched a single-payer NHI programme in 1995. The National Health Insurance Research Database (NHIRD), a medical claims database, was established and released for research purposes. The NHIRD contains all inpatient and outpatient claims data in Taiwan, including patients' demographic characteristics, dates of clinical visits, disease diagnoses, prescription medications and expenditure amounts. More than 99% of the total population of Taiwan was enrolled in the NHI programme. In this study, we analysed the claim data of one million beneficiaries randomly sampled from all of the beneficiaries registered in 2000. Patients with rheumatoid arthritis (RA) who were prescribed a SSZ for at least one month during the study period and defined as the SSZ cohort. The initial SSZ treatment date was defined the index data. We excluded patients with a history pf opioid dependence and abuse diagnosed before the index date. A control cohort was also established by randomly frequency-matching age, sex, morphine or fentanyl use and index year of the SSZ cases to RA patients from the insured population without a history of SSZ treatment.

Statistical Analysis

For animal studies, all data are given as mean±SD. Statistical analyses were performed with SPSS package (version 18.0) using unpaired Student's t test and ANOVA with Bonferroni's or Tukey's multiple comparison post hoc tests, where appropriate.

### Example 1 Knockout of System x_{c}⁻ in Mice is Able to Increases the Analgesic Effect of Opioid

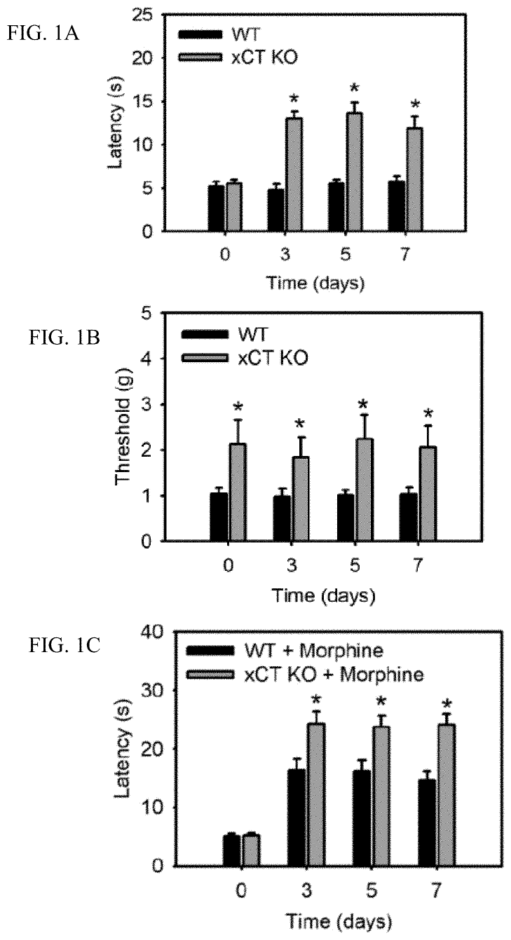

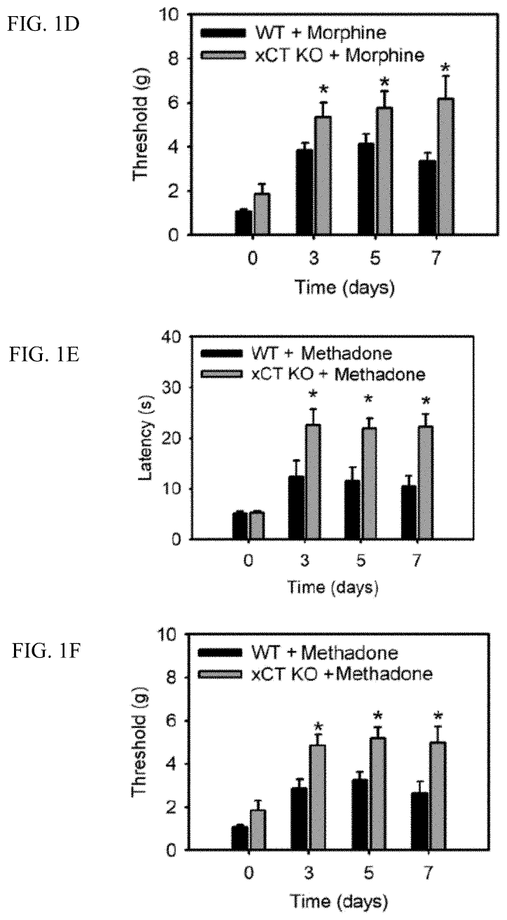

The xCT-knockout mice were utilized to study the role of system x_{c}⁻ in the morphine or methadone-mediated analgesic effect and pain relief. Wild-type (WT) and xCT-knockout (xCT KO) mice were subjected to the thermal hyperalgesia and mechanical allodynia tests. The results showed that knockout of system x_{c}⁻ in mice had more latencies of the first sign of pain in the hot-plate test and withdraw threshold in von Frey filament test compared to WT mice, indicating pain insensitivity in xCT KO mice (FIGS. 1A and 1B). Moreover, morphine or methadone also significantly increased the latency of first sign of pain and withdraw threshold in both genotypes (FIGS. 1C, 1D, 1E and 1F). The analgesic effect of morphine or methadone in xCT KO mice was higher than that in WT mice, suggesting inhibition of system x_{c}⁻ is not only to promote the effect of pain insensitivity but also enhance the analgesic effect of opioid.

### Example 2 the Pharmacological Inhibition of System x_{c}⁻ Enhances the Analgesic Effect of Opioid

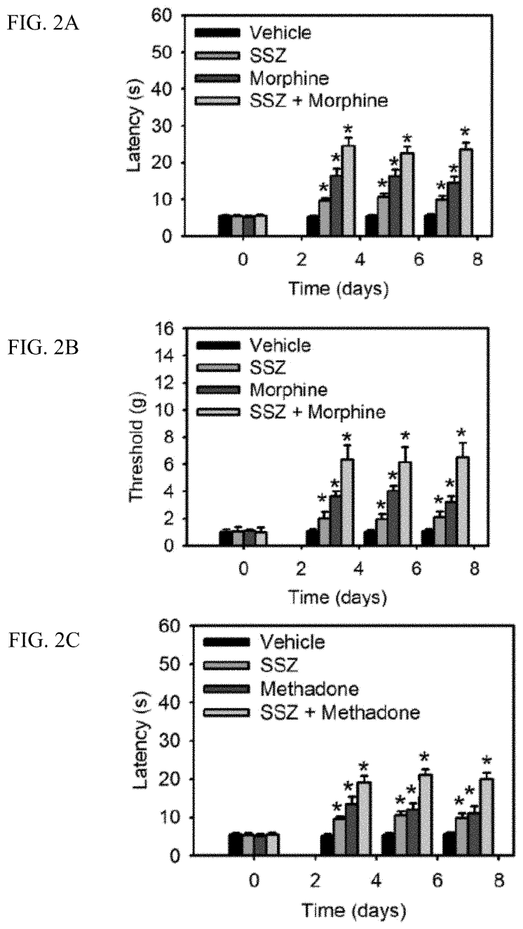

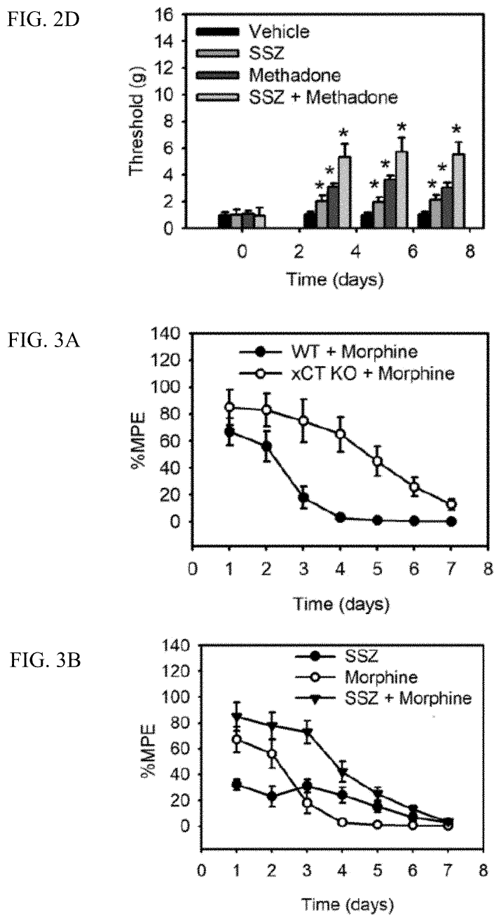

To test whether pharmacological inhibition of system x_{c}⁻ also had similar biological effects, the wild-type mice were received the co-administration of sulfasalazine (SSZ), a drug approved by the US Food and Drug Administration (FDA) that blocks system x_{c}⁻, and morphine or methadone under thermal hyperalgesia and mechanical allodynia tests. The latencies of the first sign of pain in the hot-plate test and withdraw threshold in von Frey filament test in the SSZ, morphine or methadone alone-treated mice or the combination of morphine or methadone and SSZ-treated mice were more than those in vehicle-treated mice (FIGS. 2A, 2B, 2C and 2D). Interestingly, the combination of morphine and SSZ or methadone and SSZ-treated mice showed the additive effect in analgesic effect compared to morphine, methadone or SSZ alone-treated mice. Therefore, these results show that the pharmacological inhibition of system x_{c}⁻ is able to enhance the analgesic effect for opioid.

### Example 3 the Blockage of System X_{c}⁻ Delays the Opioid Tolerance

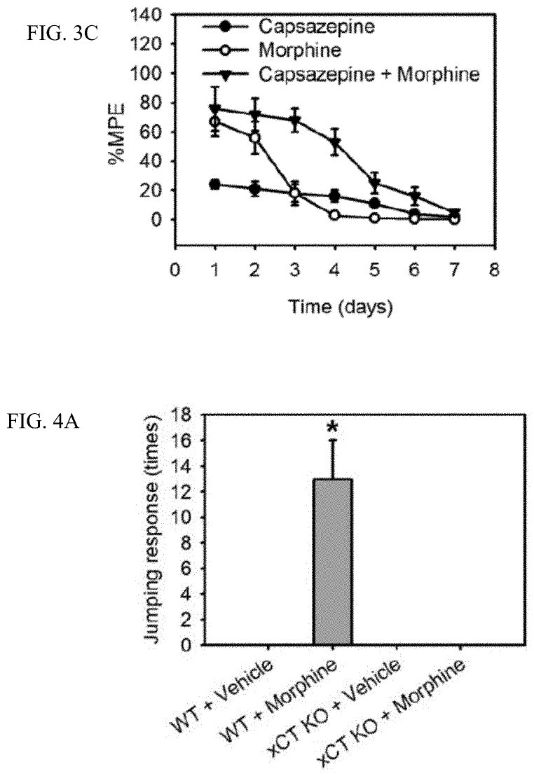

To test whether system x_{c}⁻ plays a role in opioid tolerance, WT and xCT KO mice were injected intraperitoneally with morphine twice daily and tested on 7 days continuously for determination of their respective morphine percent maximal possible effect (% MPE). Analgesic effect was expressed as % MPE, where % MPE=(test−baseline latency)/(cutoff−baseline)×100. The results showed that WT mice had significant opioid tolerance on day 3 after heavy morphine treatment (20 mg/kg/day). Interestingly, there was a significant time delay in the process of opioid tolerance in xCT KO mice (FIG. 3A). Next, the WT mice were received the co-administration of system x_{c}⁻ inhibitor, SSZ or capsazepine, and morphine twice daily for one-week observation. SSZ or capsazepine co-treatment significantly attenuated the development of tolerance and prolong the number of days of morphine-induced analgesic effect in comparison to the morphine alone group (FIGS. 3B and 3C). These results indicate that combination of system x_{c}⁻ inhibitor and opioid is able to delay the opioid tolerance.

### Example 4 the Blockage of System x_{c}⁻ Inhibits Opioid Dependence

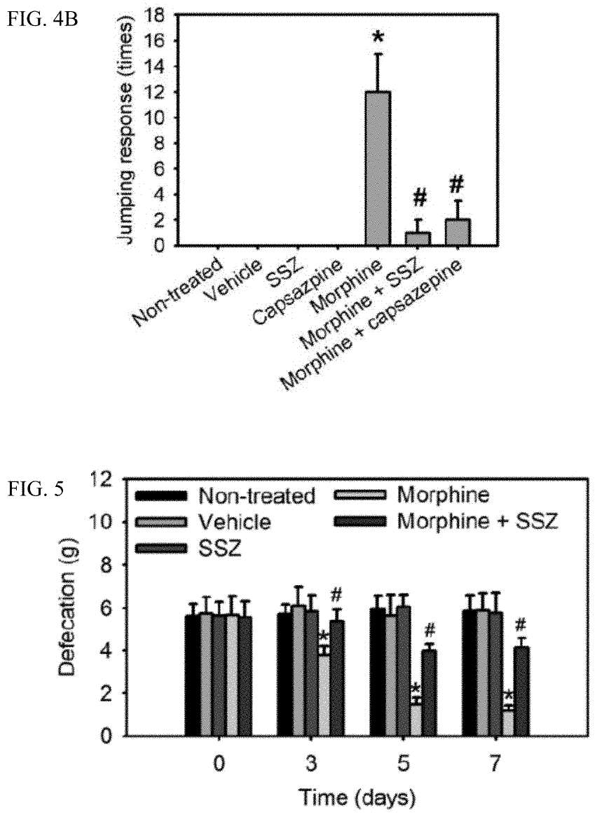

Naloxone is an opioid antagonist for opioid receptors. In opioid-dependent patients, naloxone is used in the treatment of opioid-overdose-induced respiratory depression. However, the naloxone treatment in opioid-dependent patients is able to induce acute withdrawal syndromes and therefore, it can be used to verify opioid-dependence in opioid-use patients by observing these syndromes. In the animal studies, naloxone-induce jumping behavior is a one of standards to verify whether mice have the opioid-dependence and is also a tool to study opioid and opiate withdrawal. It was first determined whether system x_{c}⁻ contributes to opioid dependence. WT and xCT KO mice were received the repeated injections for four consecutive days with an escalating dose schedule for the induction of chronic morphine dependence. Mice received morphine twice daily (morning and afternoon) for 4 days (day 1: 12 mg/kg and 25 mg/kg; day 2: 25 mg/kg and 50 mg/kg; day 3: 50 mg/kg and 75 mg/kg; day 4: 75 mg/kg and 100 mg/kg). On the testing day (day 5), a final morphine dose (50 mg/kg) was administered before naloxone-induced acute withdrawal syndromes. After naloxone (5 mg/kg) treatment, mice were observed for an hour to observe whether these mice showed any withdrawal symptoms such as restlessness, jumping behavior and incontinence. There was a significant jumping behavior and incontinence in WT mice (FIG. 4A). However, xCT KO mice didn't have such phenomenon, suggesting blockage of system x_{c}⁻ is able to prevent opioid-dependence. To observe the effect of system x_{c}⁻ inhibits in opioid withdrawal, mice with chronic morphine dependence were randomly divided into the following groups (sample size N=6 in each group): group 1: control mice without any treatment; group 2: morphine-dependent mice with vehicle treatment; group 3: morphine-dependent mice with naloxone (5 mg/kg) treatment; group 4: morphine-dependent mice with naloxone (5 mg/kg)+methadone (10 mg/kg) treatment; group 5: morphine-dependent mice with naloxone (5 mg/kg)+SSZ (30 mg/kg) treatment; and group 6: morphine-dependent mice with naloxone (5 mg/kg)+capsazepine (5 mg/kg). Our results demonstrated that morphine-dependent mice with naloxone treatment showed a significant jumping behavior compared to other groups. SSZ and capsazepine inhibited naloxone-induced jumping behavior in morphine-dependent mice (FIG. 4B). These results indicate that combination of system x_{c}⁻ inhibitor and opioid is able to inhibit the process of opioid dependence and improves the withdrawal symptoms in opioid dependency.

### Example 4 the System x_{c}⁻ Inhibitor, SSZ, Improves the Opioid-Induced Constipation

Opioid-induced constipation is a side effect for using opioid pain relief medication. We next determine whether the combination of system x_{c}⁻ inhibitor, SSZ, and opioid is able to improve opioid-induced constipation. Mice received daily morphine alone treatment for 7 days developed the opioid-induced constipation (FIG. 5). SSZ alone treatment didn't decreased or increased defecation compared to nontreated group or vehicle-treated group. Interestingly, mice with the intraperitoneal injection (IP) of morphine and SSZ combination significantly improved the opioid-induced constipation. These results indicate that the combination of system x_{c}⁻ inhibitor, SSZ, and opioid is able to suppress opioid-induced constipation.

What is claimed is:

1. A method for modulating tolerance to opiates, opioids or opioid analgesics in a subject who has developed or is at risk of developing a tolerance for the opioid or analgesic, comprising administering a therapeutically effective amount, or a sub-therapeutically effective amount, of opiate, opioid or opioid analgesic and a therapeutically effective amount of a cystine/glutamate antiporter system x_{c}⁻ inhibitor.

2. The method of claim 1, wherein the modulating tolerance includes delaying, reducing, relieving, attenuating, and/or reversing tolerance to opioids or opioid analgesics.

3. The method of claim 1, wherein the subject obtains therapeutic effect from a lower dose of the opiate, opioid or opioid analgesic than before modulation of tolerance. In one aspect, the modulated subject obtains an improved therapeutic effect from the same dose of the opioid or opioid analgesic compared to before modulation.

4. The method of claim 1, wherein the subject exhibits opiate, opioid or opioid analgesic tolerance prior to said administration and reduced opioid or opioid analgesic tolerance following said administration.

5. A method for treating acute and chronic pain and/or decreasing opiate or opioid-induced side effects in a subject in a need of opiate, opioid or opioid analgesic therapy, comprising administering a therapeutically effective amount, or a sub-therapeutically effective amount, of opiate, opioid or opioid analgesic and a therapeutically effective amount of a cystine/glutamate antiporter system x_{c}⁻ inhibitor.

6. The method of claim 5, wherein the cystine/glutamate antiporter system xc-inhibitor enhances the analgesic effect of opiate, opioid or opioid analgesic.

7. The method of claim 5, wherein the opiate or opioid-induced side effect is sedation, dizziness, bowel dysfunction, nausea, vomiting, somnolence, physical dependence, tolerance, addiction, respiratory depression, headache, dry mouth, sweats, asthenia, hypotension, dysphoria, delirium, miosis, pruritis, urticaria, urinary retention, hyperalgesia, allodynia or a combination thereof.

8. The method of claim 7, wherein the bowel dysfunction is constipation, decreased gastric emptying, abdominal cramping, spasm, bloating, delayed gastro-intestinal transit or formation of hard dry stools.

9. The method of claim 7, wherein the bowel dysfunction is constipation.

10. The method of any of claims 1 to 9, wherein the cystine/glutamate antiporter system xc-inhibitor is administered concurrently or separately with an opiate, opioid or opioid analgesic.

11. The method of any of claims 1 to 10, wherein the amount of the opiate, opioid or opioid analgesic can be reduced when administering concurrently or separately with the cystine/glutamate antiporter system xc-inhibitor.

12. The method of any of claims 1 to 11, wherein during the concurrent administration, the amount of the opiate, opioid or opioid analgesic is reduced.

13. The method of any of claims 1 to 12, wherein the therapeutically effective amount of opiate, opioid or opioid analgesic is the dosage used clinically.

14. The method of any of claims 1 to 13, wherein the sub-therapeutically effective amount of opiate, opioid or opioid analgesic is the dosage under that used clinically

15. The method of any of claims 1 to 14, wherein the therapeutically effective amount of a cystine/glutamate antiporter system x_{c}⁻ inhibitor is the dosage used clinically.

16. The method of any of claims 1 to 15, wherein the therapeutically effective amount of sulfasalazine ranges from about 0.75 mg/kg to about 28.57 mg/kg.

17. A combination comprising a therapeutically effective amount, or a sub-therapeutically effective amount, of opiate, opioid or opioid analgesic and a therapeutically effective amount of a cystine/glutamate antiporter system x_{c}⁻ inhibitor.

18. The combination of claim 17, wherein the opiate, opioid or opioid analgesic and the cystine/glutamate antiporter system x_{c}⁻ inhibitor are contained in a medicament or the opiate, opioid or opioid analgesic and the cystine/glutamate antiporter system x_{c}⁻ inhibitor are each independently contained in a separate medicament.

19. The method of any of claims 1 to 16 or a combination of claim 17 or 18, wherein the opiate, opioid or opioid analgesic is opiate, opioid, codeine, fentynal, hydrocodone, hydromorphone, buprenorphine, thebaine, meperidine, methadone, morphine, oxycodone, oxycodone, acetaminophen, oxycodone and naloxone, heroin, heroin laced with fetynal, pethidine, opium, NKTR-181, Difelikefalin, tramadol, tapentadol, levorphanol, sufentanil, pentazocine, oxymorphone or a combination thereof.

20. The method of any of claims 1 to 16 or a combination of claim 17 or 18, wherein the cystine/glutamate antiporter system x_{c}⁻ inhibitor is sorafenib, regorafenib, sulfasalazine, 2-hydroxy-5-((4-(N-pyridin-2-ylsulfamoyl)phenyl)ethynyl)benzoic acid, 5-aminosalicylic acid (5-ASA), sulfapyridine (SP), erastin, L-glutamate, L-cystine, L-alpha-aminoadipate, L-alpha-aminopimelate, L-homocysteate, L-b-N-oxalyl-L-a,b-diaminopropionate (beta-L-ODAP), L-alanosine, ibotenate, L-serine-O-sulphate, (RS)-4-bromohomoibotenate, quisqualate, (S)-4-carboxyphenylglycine, RS-4-Br-Homo-IBO, 2-amino-3-(5-methyl-3-oxo-1,2-oxazol-4-yl)propanoic acid (AMPA), arachidonylcyclopropylamide (ACPA), N-acetylamino-3-chloro-N-(2-diethylaminoethyl)benzamide (NACPA), TFMIH, NEIH, (S)-4-carboxyphenyglycine (4-S-CPG), 4-S-SPG, TSA, CPPG, capsazepine or any combination thereof.

21. The method of any of claims 1 to 16 or a combination of claim 17 or 18, wherein the cystine/glutamate antiporter system x_{c}⁻ inhibitor is sorafenib, regorafenib, sulfasalazine or capsazepine or a combination thereof.

22. The method of any of claims 1 to 16 or a combination of claim 17 or 18, wherein the cystine/glutamate antiporter system x_{c}⁻ inhibitor is sulfasalazine or capsazepine.
